(19) United States
(12) Patent Application Publication
(10) Pub. No.: US 2021/0158721 A1
(43) Pub. Date: May 27, 2021

(54) MEASURING COGNITION AND DETECTING COGNITION IMPAIRMENT

(71) Applicant: Predictor Systems, LLC, Seattle, WA (US)

(72) Inventors: Ovidiu Lucian STAVRICA, Seattle, WA (US); Dirk Duncan EIDE, Spokane, WA (US)

(21) Appl. No.: 17/102,336

(22) Filed: Nov. 23, 2020

Related U.S. Application Data
(60) Provisional application No. 62/941,552, filed on Nov. 27, 2019.

(51) Int. Cl. G09B 19/00; G16H 10/20; G16H 50/70; G06K 19/06; G06F 21/31; G06F 3/0488

(52) U.S. Cl. CPC G09B 19/00; G16H 10/20; G16H 50/70; G06F 3/04817; G06K 19/06112; G06F 21/31; G06F 3/04886; G06K 19/06037

(57) ABSTRACT

The present disclosure is directed to a cognitive evaluation system that includes a display device, a plurality of user input actuators, and a computer-readable storage medium having instructions stored thereon that, when executed by the one or more processors. The processor cause the system to provide a plurality of cognitive evaluation tests to one or more users over a first period of time, determine baseline cognitive evaluation information for at least one user of the one or more users based at least in part on test results from the plurality of cognitive evaluation tests, administer one or more additional cognitive evaluation tests to the at least one user via the display and the plurality of user input actuators, and identify, based at least in part on test results from the one or more additional cognitive evaluation tests, a cognitive impairment condition of the at least one user.

### A. SUMMARY OF THE INVENTION

Techniques presented herein are generally directed to a cognition evaluation (“CE”) system for identifying various types of cognition impairments affecting one or more users, such as by comparison of current user test performance with baseline information generated by the CE system based on previous test performance, either by the current user or others. In certain embodiments, such a CE system may be considered to include two parts: a client test environment, such as an Internet-connected multi-touch client computing device (e.g., a computer tablet) for presentation of an interactive testing experience to a user; and one or more remote computing servers, such as for processing interaction timeline data collected by the client computing device or other functionality.

### B. BRIEF DESCRIPTION OF THE DRAWINGS

FIG. 1 depicts an exemplary element layout of an application log-in screen. In particular, all interactive elements are located in the upper half of the screen to prevent being obscured when the on-screen keyboard is presented by the device.

FIG. 2 identifies the location where the client name is displayed on the application log-in screen. This name may be the university, college or sport team name where the user is playing, or the company name where the user is employed.

FIG. 3 identifies the location where the application software version or equivalent technical information is displayed.

FIG. 4 identifies the location where the unique hardware device identifier or equivalent technical information is displayed.

FIG. 5 shows the QR code scanner camera presentation when the user chooses to authenticate using their QR code. The camera region shows a live camera view. The screen region outside this camera view is black.

FIG. 6 identifies the camera toggle button. Touching this button element causes the application to switch between the front camera and the back camera views; the text on this button element also toggles between BACK CAMERA and FRONT CAMERA, as appropriate.

FIG. 7 identifies the cancel button. Touching this button element will cause the QR code scanner to close and return the user back to the FIG. 1 log-in screen.

FIG. 8 identifies the screen region that contains the test instructions.

FIG. 9 identifies the available tile positions for displaying the left-hand arming icons.

FIG. 10 identifies the available tile positions for displaying the right-hand arming icons.

FIG. 11 shows a sample placement of the left-hand arming icons that the user may touch to initiate test arming sequence.

FIG. 12 shows a sample placement of the right-hand arming icons that the user may touch to initiate test arming sequence.

FIG. 13 shows a sample placement of the left-hand eye icons that the user may touch and stare at to initiate test arming sequence.

FIG. 14 shows a sample placement of the right-hand eye icons that the user may touch and stare at to initiate test arming sequence.

FIG. 15 shows sample placement of the original arming icon locations where the user is required to maintain finger contact with the tablet screen.

FIG. 16 identifies the available positions for displaying the test target image.

FIG. 17 identifies the available positions for displaying one or more matching images along with one or more non-matching images.

FIG. 18 is a flowchart representation of a polling “touch start” tile selection debounce algorithm.

FIG. 19 is a flowchart representation of a polling “touch end” tile selection debounce algorithm.

FIG. 20 shows a sample dataset for the cognition evaluation session timeline.

FIG. 21 shows a user looking at a tablet that is positioned too close to the face.

FIG. 22 indicates the field of view angle of the tablet screen as seen by the user in FIG. 21.

FIG. 23 shows a user looking at a tablet that is positioned an acceptable distance from the face.

FIG. 24 indicates the field of view angle of the tablet screen as seen by the user in FIG. 23.

FIG. 25 is a flowchart representation of the cognition evaluation session flow.

FIG. 26 shows a sample cognition classification matrix that identifies 4 cognitive processing areas: left brain motor control, right brain motor control, lingual processing and spatial processing.

FIG. 27 shows a sample test class from the classification matrix defined by FIG. 26 with associated weights that indicate the user may utilize significant left brain motor control and lingual processing to complete.

FIG. 28 shows a sample test class from the classification matrix defined by FIG. 26 with associated weights that indicate the user may utilize significant left brain motor control and spatial processing to complete.

FIG. 29 shows a sample test class from the classification matrix defined by FIG. 26 with associated weights that indicate the user may utilize significant right brain motor control and mixed lingual/spatial processing to complete.

FIG. 30 shows a sample test class from the classification matrix defined by FIG. 26 with associated weights that indicate the user may utilize significant right brain motor control and lingual processing to complete.

FIG. 31 shows a sample test class from the classification matrix defined by FIG. 26 with associated weights that indicate the user may utilize significant right brain motor control and spatial processing to complete.

FIG. 32 identifies a specific target image tile position from among the FIG. 16 tile locations.

FIG. 33 identifies the tile position from the FIG. 17 tile locations that always contains the complementary image to the target image in FIG. 32.

FIG. 34 identifies the tile positions from the FIG. 17 tile locations that are always populated with one or more images.

### C. DETAILED DESCRIPTION

In the following description, certain details are set forth in order to provide a thorough understanding of various embodiments of devices, systems, methods and articles. However, one of skill in the art will understand that other embodiments may be practiced without these details. In other instances, well-known structures and methods associated with, for example, circuits, such as transistors, integrated circuits, logic gates, memories, interfaces, bus systems, etc., have not been shown or described in detail in some figures to avoid unnecessarily obscuring descriptions of the embodiments.

Unless the context requires otherwise, throughout the specification and claims which follow, the word “comprise” and variations thereof, such as “comprising,” and “comprises,” are to be construed in an open, inclusive sense, that is, as “including, but not limited to.” Reference to “at least one of” shall be construed to mean either or both the disjunctive and the inclusive, unless the context indicates otherwise.

Reference throughout this specification to “one embodiment,” or “an embodiment” means that a particular feature, structure or characteristic described in connection with the embodiment is included in at least one embodiment. Thus, the appearances of the phrases “in one embodiment,” or “in an embodiment” in various places throughout this specification are not necessarily referring to the same embodiment, or to all embodiments. Furthermore, the particular features, structures, or characteristics may be combined in any suitable manner in one or more embodiments to obtain further embodiments.

The headings are provided for convenience only, and do not interpret the scope or meaning of this disclosure.

The sizes and relative positions of elements in the drawings are not necessarily drawn to scale. For example, the shapes of various elements and angles may not be drawn to scale, and some of these elements may be enlarged and positioned to improve drawing legibility. Further, the particular shapes of the elements as drawn are not necessarily intended to convey any information regarding the actual shape of particular elements, and have been selected solely for ease of recognition in the drawings.

As utilized herein, a client computing device may be interchangeably referenced as a “tablet,” “computer tablet,” or “tablet test environment” for clarity; it will be appreciated that a variety of client computing devices may be utilized in various embodiments of techniques described herein. Similarly, reference herein to a “server” or “remote server” may be understood to describe, in various embodiments, one or more remote computing servers, each of which may provide similar or disparate functionality. Moreover, reference herein to operations performed by a cognition evaluation system may, in certain embodiments, refer to operations performed by the client computing device, the remote server, or a combination thereof.

In a typical use scenario related to at least one embodiment of techniques described herein, a user may interact with a tablet test environment on an ongoing basis (up to and including a span of multiple years) at regular or semi-regular intervals. To begin a cognition evaluation session in this exemplary embodiment, the tablet test environment may authenticate the user via a cognition evaluation application being executed by one or more processors of the computer tablet, and presented to the user via a touch display component (“screen” or “touch screen”) of the computer tablet (which may, for example, be integrated with the computer tablet in a single housing or communicatively coupled thereto). The CE system may then retrieve information regarding one or more previous sessions of the user (such as from the remote server) and invite the user to initiate one or more tests presented by the tablet test environment. Upon the completion of the specified tests, the CE system ends the current session and submits information regarding the current testing session (e.g., coordinate information, timeline information, or other information) to the remote server, such as for analysis and/or storage.

In certain embodiments, as a plurality of users complete multiple cognition evaluation sessions over time, the CE system may perform a variety of operations in order to analyze stored information regarding testing sessions for one or more respective users of the plurality of users, as well as to generate baseline profiles for each respective user. In this manner, accuracy of the CE system may improve with respect to subsequent interactions with those users.

In at least some embodiments, the CE system may perform various operations to detect a cognition impairment event. For example, the CE system may compare various information regarding a current testing session for a particular user with baseline profile information, such as baseline profile information associated with the same user or one or more other users. In certain embodiments, the CE system may identify a cognition impairment event based at least in part on baseline profile information regarding one or more users determined to share similarities with a current individual user, such as (as non-limiting examples) demographic similarities, professional similarities, geographic similarities, etc. If the recent interaction results diverge from the selected baseline (such as if differences between information regarding the current session and the selected baseline profile meet or exceed a defined threshold), the CE system may determine that the user's cognition is considered to be impaired.

This disclosure describes certain exemplary embodiments of an interactive testing interface, such as may be presented for one or more users via a CE application executing on a tablet device. It will be appreciated that in various other embodiments, one or more elements of the described interactive testing interface may vary from those described without diverging from the techniques presented herein.

In certain embodiments, the CE system may provide the user with a specific set of written and/or image-based instructions. The CE system may then present a plurality of visual and/or auditory stimuli designed to elicit specific responses from the user in accordance with the provided instructions. For example, a CE application may detect and/or record one or more user touch interactions with the touch screen, as well as one or more eye and head movements identified via an imaging component (e.g., a front-facing camera) for some or all of an interactive testing session.

Each authenticated CE session may include one or more iterations of the following generalized steps:
1. Display Test Instructions
2. Display Test Arming sequence
3. Test interaction(s)

In at least some embodiments, displaying test instructions may include providing details regarding user actions to perform the arming sequence as well as to complete the one or more subsequent test interactions. As used herein, an “arming sequence” refers to one or more user interactions with the CE system intended to calibrate and/or otherwise initialize the testing environment for a particular authenticated user, including (in certain scenarios and embodiments) to prepare the user for initiation of a single test or testing session.

One embodiment of the arming sequence requires the user to simultaneously touch a plurality of specified locations along the left and right edges of the tablet screen for a set period of time.

Likewise, one embodiment of user testing interactions populates the screen with a plurality of similarly sized image tiles, dimensioned to fit within a two-dimensional grid.

In this exemplary embodiment, the user may first identify and touch a target image from a plurality of images presented along the top and/or bottom edges of the tablet screen. The target image may be presented in a manner visually distinct from presentation of the other images along the top and bottom edges. The target image is removed from the screen when touched. The user may then locate and touch a matching image from among a plurality of images being displayed in the center area of the screen. Completing the image match action concludes one test iteration.

### 1. User Authentication

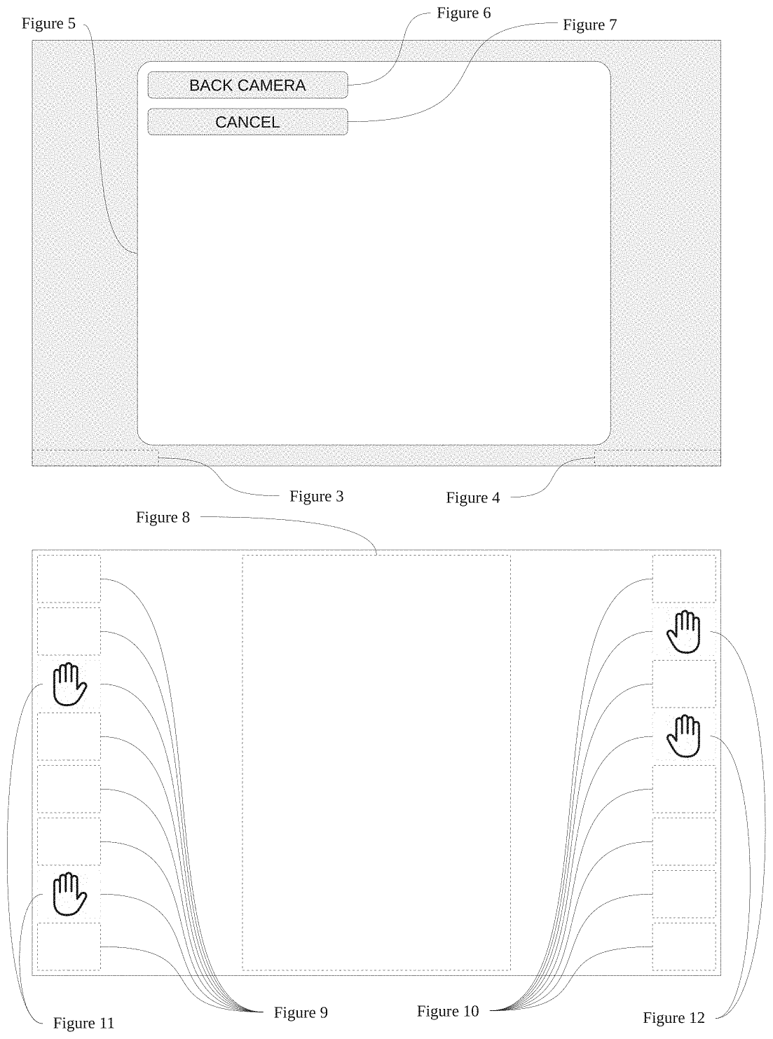

In certain embodiments, a CE application log-in screen enables a user to verify their identity and displays some additional details for identification and diagnostic purposes. In the depicted embodiment, FIG. 2 indicates an interface element for displaying a client name, such as may identify an organization or other entity associated with the CE system, the user, and/or the computer tablet; FIG. 3 indicates an interface element for displaying the software revision number, and FIG. 4 indicates an interface element for displaying a unique installation identifier for the executing CE application. It will be appreciated that one or more elements of the depicted user interface may vary in accordance with one or more alternative embodiments.

In various embodiments, the CE system may authenticate one or more users using established credentials (e.g., username, password, or other credentials) or by other method. For example, in certain embodiments a user may scan a QR code containing one or more unique identifiers.

When authenticating via QR code, the CE application may display the tablet's camera view, shown in FIG. 5, to facilitate the scanning process. The user has the ability to switch between the tablet's front and back cameras by tapping FIG. 6 or dismiss the QR scanner by touching FIG. 7. Both FIG. 3 and FIG. 4 remain visible in the QR scanner view.

The tablet application's ability to identify users by QR code facilitates a variety use modes intended to minimize user engagement time and effort. For example, an athlete user may scan a QR code sticker located on the back of their football helmet. Alternatively, a user may display a dynamically generated QR code on their smart phone via a mobile web browser. A QR code may contain either a static identifier associated with the respective user or the user's encrypted record identifier. The QR code may also include auxiliary encrypted details, including: a valid-from timestamp, an expiration timestamp, a code revision identifier, a client identifier, and other short alphanumeric or binary details. In certain embodiments, administrative personnel associated with an organization administering a cognitive evaluation session may access one or more authentication QR codes associated with users in their charge. This permits coaches to evaluate players in situ by retrieving a player's QR code via their administrative mobile web portal.

In certain embodiments, user authentication credentials such as username/password combinations and QR code data may be transmitted to a remote CE system server for verification. Username and password values may be hashed and salted via the local CE application before transmission to the remote server for enhanced user anonymity.

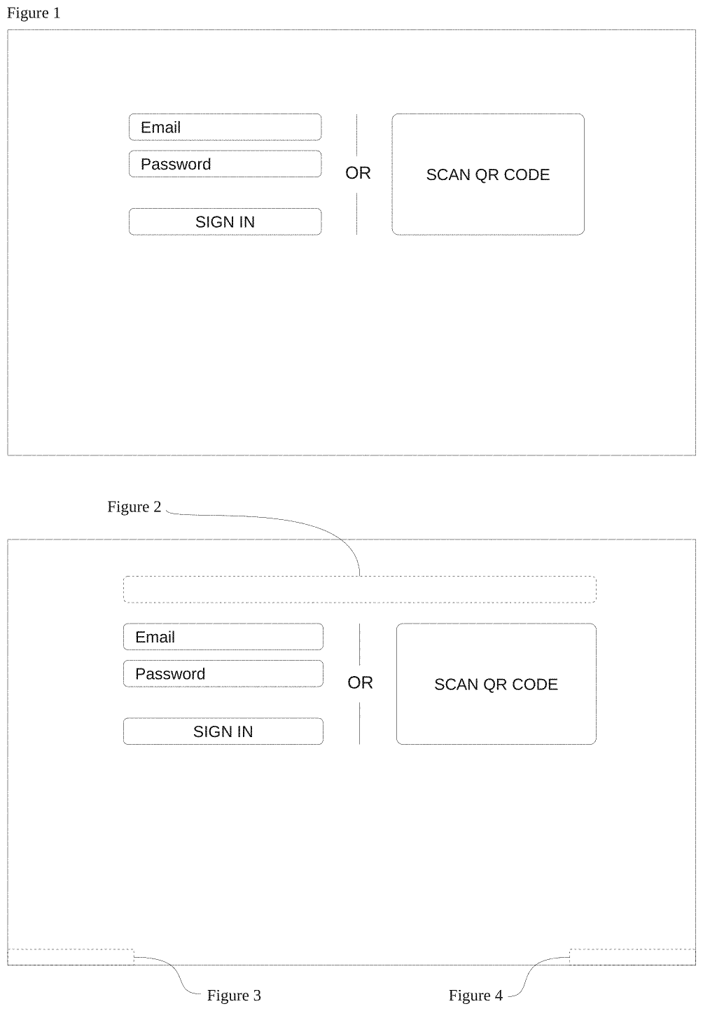

If incorrect credentials are provided for email or password fields, the application may provide one or more types of notifications, such as audio, visual, or haptic feedback to the user. As one example, the CE system may visually present the displayed elements in FIG. 1 in a stylized manner to indicate failure, such as by animating a vibration or specialized coloring of those elements. As another example, if an invalid or expired QR code is presented to the camera, the CE application may revert the user interface back to the FIG. 1 log-in screen and gently shake the elements on the screen to indicate failure. It will be appreciated that any manner of notification may be utilized to inform the user that their authentication credentials have not been accepted by the CE system.

In the accompanying figures, the depicted user interface is intended in black and white for clarity. However, in various embodiments the screen background, instruction text, instruction images and arming icons may utilize various other color schema, such as may be implemented by the CE system in accordance with one or more client and/or user preferences. In addition, individual authentication operations, arming operations, and testing operations provided by the CE system with respect to one or more users may employ varied color schema, either in a single testing session or between such sessions.

In at least one embodiment, the computer tablet may be placed on an immovable surface to prevent sliding, rocking, or other forms movement from interfering with or otherwise affecting user interaction with the tablet.

### 2. Test Instructions and Arming Sequence

In certain embodiments, following user authentication the CE application may display the instruction and arming sequence screen. The following description is provided regarding one embodiment depicted via illustrations corresponding to FIGS. 8-17; it will be appreciated that multiple variations of the described operations and user interface may be used by embodiments of the CE system, including variations regarding the location of particular elements within the described user interface; display operations of the CE system with respect to that user interface; timing of described operations; etc.

In the depicted embodiment, arming and testing instructions are displayed in the area indicated by FIG. 8, and may include words, images, or some combination thereof. The instructions may be static or interactive, such as by allowing the user to interact with content by tapping, swiping, scrolling or other gestures.

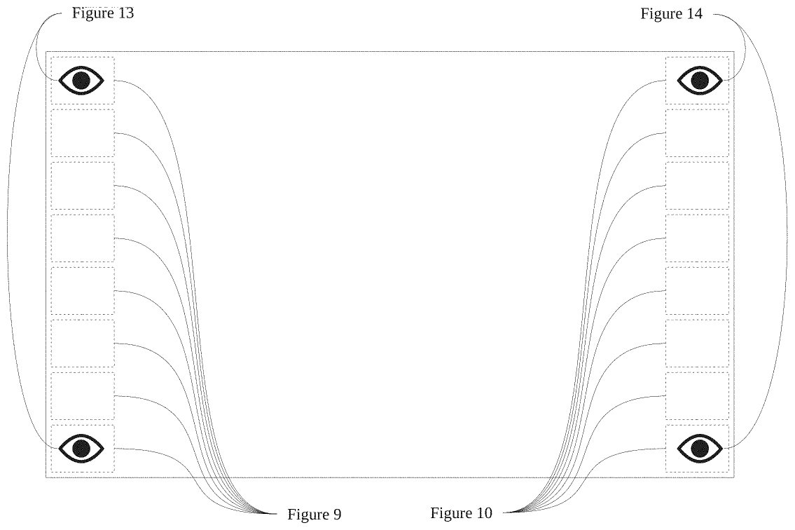

One embodiment of the arming sequence displays four arming icons as two hand images on each side the screen. The left and right tile positions, respectively labelled FIG. 9 and FIG. 10, indicate possible screen locations in which the arming icons, FIG. 11 and FIG. 12, may be displayed. The CE application may, in certain embodiments, vary the location of the arming icons to prevent the user from developing cognitive or muscle-memory familiarity with the arming sequence associated with multiple tests and/or testing sessions.

In at least the depicted embodiment, the user may touch and hold the arming icons for a short duration (e.g., three seconds) to initiate a test. Upon being touched, each arming icon may be cleared from the respective tile to provide the user with visual confirmation. As the arming sequence engages, the instructions in FIG. 8 are removed from the screen and all tile positions in FIG. 11 and FIG. 12 flash white 3 times, once per second, with a timing rate of 500 ms as white and 500 ms as black. The white to black timing ratio may vary, to prevent cognitive familiarity with the arming sequence. For example, an alternate white to black timing rate may be 250 ms and 750 ms respectively. Likewise, the arming time period may be increased or decreased between test iterations; and the exact arming duration may or may not be documented in the FIG. 8 instructions, again as needed by each particular test.

During the arming sequence, one of the tile columns, either FIG. 11 or FIG. 12 flashes brighter than the other, to designate which hand the user is to use for interacting with the test. Alternatively, only the right hand or left hand tile column may flash, again to designate the hand the user is required use for interacting with the test.

In at least one embodiment, the CE system may monitor one or more sensors of the computer tablet (e.g., accelerometer and gyroscope sensors) to ensure that the tablet does not experience any movement that may interfere with accurate assessment of the test results. In such an embodiment, the CE system may pause or terminate the arming sequence if movement exceeding configured thresholds is reported by the hardware sensors.

In addition, in certain embodiments the CE system may determine to terminate and/or reset the arming sequence in response to detecting one or more of the following conditions:
1. User removes a finger from a tile that contains an arming icon
2. User slides finger out of tile area that contains an arming icon
3. User finger touches a tile position in FIG. 11 or FIG. 12 that has no arming icon
4. User repositions, tilts or otherwise moves the tablet.

In the event the arming sequence is interrupted, the CE system may determine to redisplay the instructions of FIG. 8 as a visual cue. In addition, in certain embodiments the display positions of the arming icons indicated by FIG. 11 and FIG. 12 may remain the same or may change, as may be configured for each particular test.

In an alternative embodiment of the arming sequence, the CE system may require the user to touch one of the four ‘eye’ icons indicated by FIG. 13 and FIG. 14, stare at it for a short duration (e.g., two seconds), and then touch it again. The user then repeats a similar touch-stare-touch sequence for the remaining three ‘eye’ icons indicated by FIG. 13 and FIG. 14. While the user does this, the CE application may track one or more aspects of the user's face and eye movement (including in certain embodiments to store a video recording or other captured information regarding such movement) along with timestamp information regarding user interaction with each of the four ‘eye’ icons. The CE tablet application may transmit some or all of such information to the remote server for analysis and/or storage, such as for offline eye-tracking calibration to correlate recorded eye movement in subsequent tests.

Variations in this and other embodiments may allow for changes in the number and location of eye icons, icon tapping routines, and the period of time required for the user to stare at each icon. In certain embodiments, the CE system may capture complementary touch events during one or more portions of the arming sequence in order to effectively calibrate eye-tracking operations regarding each testing session and/or respective user. As indicated elsewhere herein, cumulative calibration data for each user may increase the baseline accuracy and consequent eye-tracking sensitivity for that user or other users.

### 3. Test Interaction

In various embodiments, the CE system may display a testing screen upon completion of the arming sequence.

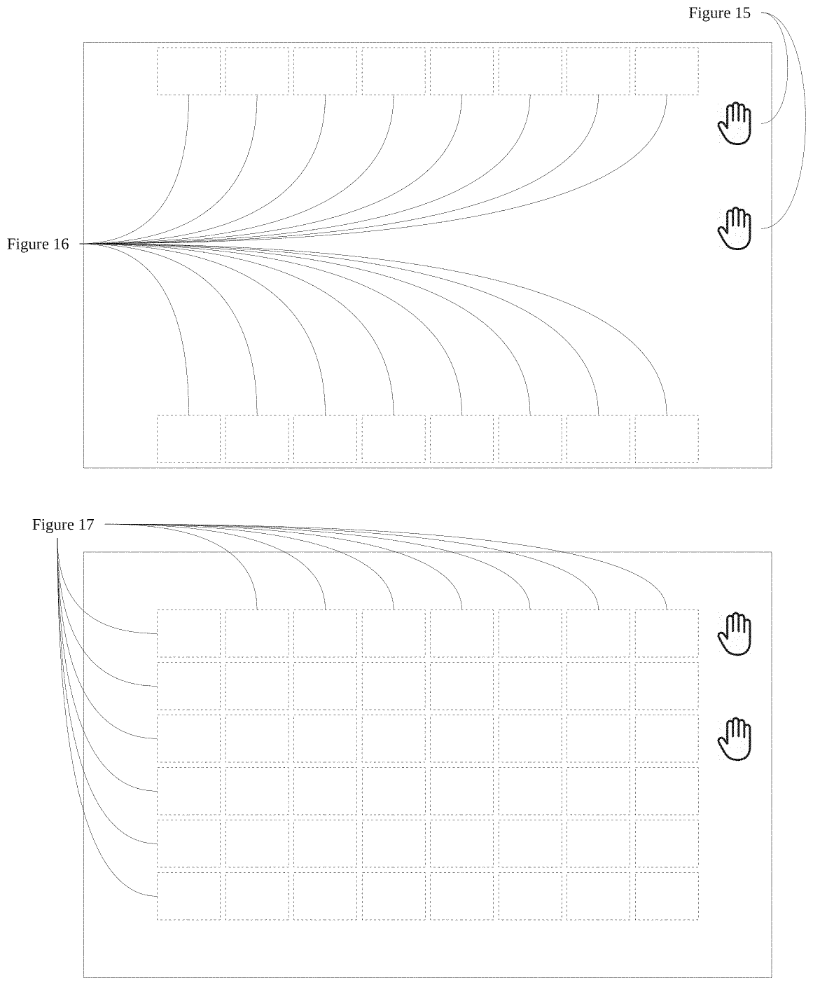

In the depicted exemplary embodiment of the test interaction, the user may utilize only a designated hand to interact with icons in indicated tile locations, such as those indicated by FIG. 16 and FIG. 17. To effectuate such a limitation, the CE system may require the user to continue touching the original arming icon tile locations with the non-designated hand. The non-designated hand arming icons may be displayed on the screen for a portion or entirety of the test, as shown in the FIG. 15 example. The displayed arming icons provide a cue to the user to continue touching the screen at those locations.

By continuing to touching the arming icons with the non-designated hand, the user is forced to employ the designated hand to complete the test. Among other things, the ability to enforce the designated hand enables the test to collect biometrics on one or more targeted brain locations (such as a right or left hemisphere) while forcing the player to complete the test while at least nominally multitasking.

To complete the test, the CE system may require the user to perform certain operations within a defined time limit. As one non-limiting example, the user may be required to perform the following operations as quickly as possible:
1. Select the target image from among the tile positions identified in FIG. 16
   2. Select a matching image from among the tile positions identified in FIG. 17

In one embodiment of the test, the tiles in FIG. 16 are populated with a series of images. One of the images is displayed in a manner that makes it visually distinct from the other images. For example, all the images may be dimmed, except for one image which is shown at full brightness. Another example involves showing the images in grayscale, except for one which is shown in color. In yet another example, the image content of one image may set it apart from the other images, such as one banana image being shown in a group of various images of apples. As with other specific testing elements described herein, it will be appreciated that various embodiments may use one or more combinations or variations of such elements without departing from the presented techniques. As non-limiting examples, in certain embodiments FIG. 16 tile positions may contain content other than images; may include more than one unique target image; and/or may display the unique target image more than once or in more than one location.

To select the target image, the user may touch the image with the designated hand until the image clears from the screen. The CE system may, for example, clear the target image shortly after (e.g., 25 milliseconds after) it is touched by the user. In certain embodiments, the exact time for such clearing of the target image may be modified in each test's parameters. Once the CE system clears the target image, it may permit the user to lift their finger off the screen. If the user lifts their finger from the target image before the required touch time elapses, the touch timer may reset, in which case the CE system may require the user to repeat the selection process.

After selecting the target image, the user may find and select the matching image with the designated hand from a plurality of images located in tiles identified by FIG. 17. Not all FIG. 17 tile positions are required to contain images. As well, a test may be configured to display more than one valid matching image.

In at least one test embodiment, the user may be required to select the target image and then to select a complementary image that is related in some way to the target image. For example, the target image may contain a banana and the complementary image may contain a pear, while the remaining displayed images may contain various types of automobiles. In another example, the target image may contain a simple arithmetic problem, “2+2”, and the complementary image contains the answer “4” while other image candidates contain other numbers. The CE system may utilize a variety of such relationships and images during the testing process.

In a variation of the preceding embodiment, a plurality of target images may be displayed on the screen, in either the top, bottom, or both FIG. 16 rows. Some or all tile locations indicated by FIG. 17 may contain numbers, words and general images, including one tile location that contains a number which corresponds to the number of target images identified in the FIG. 16 locations. The user may select all the target images, and then select the number tile from among FIG. 17 locations that corresponds to the target image count.

In another embodiment, the arming sequence may be integrated directly into the test itself. The arming icons themselves may be replaced with specific images. Once arming is complete, the arming images are removed from the screen, and the FIG. 17 tile coordinates are partially or fully populated with a plurality of images that include at least one image that corresponds with one or more of the arming images.

In certain embodiments, the CE system may determine to pause and/or terminate a test or testing session responsive to identifying one or more of the following conditions:
1. User removes finger on the non-designated hand from one of the displayed arming icon tile locations
2. User slides a finger on the non-designated hand out of tile area that displays one of the arming icons
3. User touches the screen anywhere other than the target image before selecting the target image from among the locations indicated by FIG. 16
4. User touches the screen anywhere other than the matching image after selecting the target image from among the locations indicated by FIG. 16
5. The maximum allowed test time expires, as configured for each test

In an embodiment, the CE system may determine not to present a terminated test to a user during the same testing session. An interrupted arming sequence may not constitute an aborted test, as the test would not yet have been revealed to the user.

### 4. Detecting Test Image Selection

In at least one embodiment, the CE system may employ one or more debounce algorithms to ensure that information captured regarding testing operations include only user-intended interface interactions, such as by requiring the user to touch a given tile coordinate for an extended period of time before the touch action is accepted. Such debounce algorithms may also reduce the collection by the CE system of spurious data events that may be generated by touch detection components of the tablet test environment.

In the exemplary embodiment, the CE system may utilize such a debounce algorithm to capture timestamp information regarding an initial “touch start” event when the user initially touches the screen, but may determine not to capture or act on that event until a minimum time span has elapsed. The debounce time span may be constant, or may be a configurable parameter associated with a specific test, user, or client.

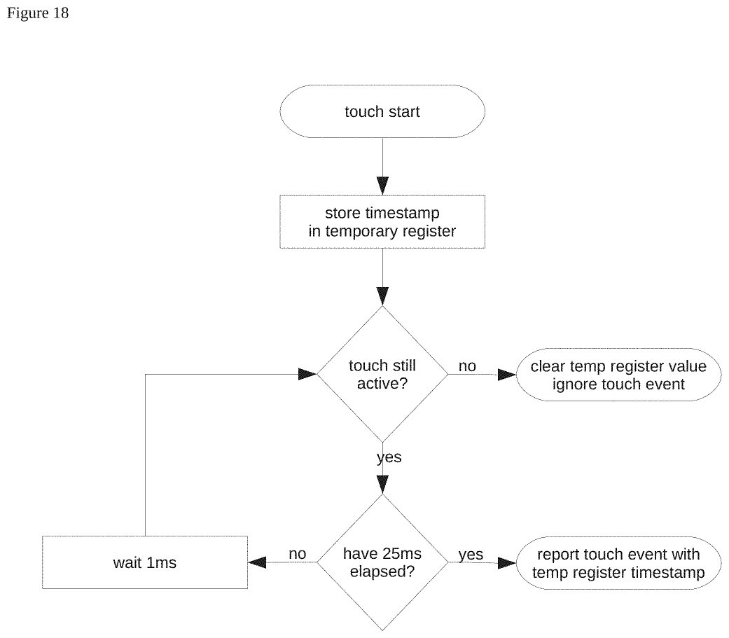

The FIG. 18 flowchart depicts an exemplary operational flow of a polling-based debounce algorithm implemented in one embodiment. In another embodiment, hardware processing requirements may be minimized by utilizing an interrupt-based debounce algorithm, such as may be customized in accordance with one or more associated programming languages.

In the depicted embodiment, a debounce algorithm is applied independently to every tile position on the test screen, including tile locations identified in FIG. 9, FIG. 10, FIG. 16, and FIG. 17. Debounce processing performed on a tile coordinate is independent of the debounce processing performed on any other tile.

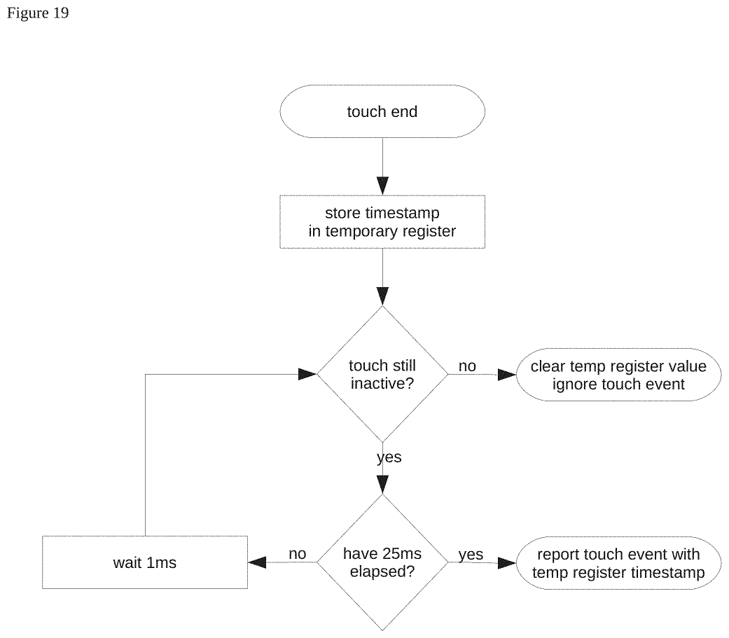

The CE system may, in certain embodiments, utilize one or more debounce algorithms to detect when the user ends contact with the screen. As shown in FIG. 19, it captures the timestamp of an initial “touch end” event, but does not report that event to one or more portions of the CE application until a minimum time span has elapsed.

### 5. Session Timeline Dataset

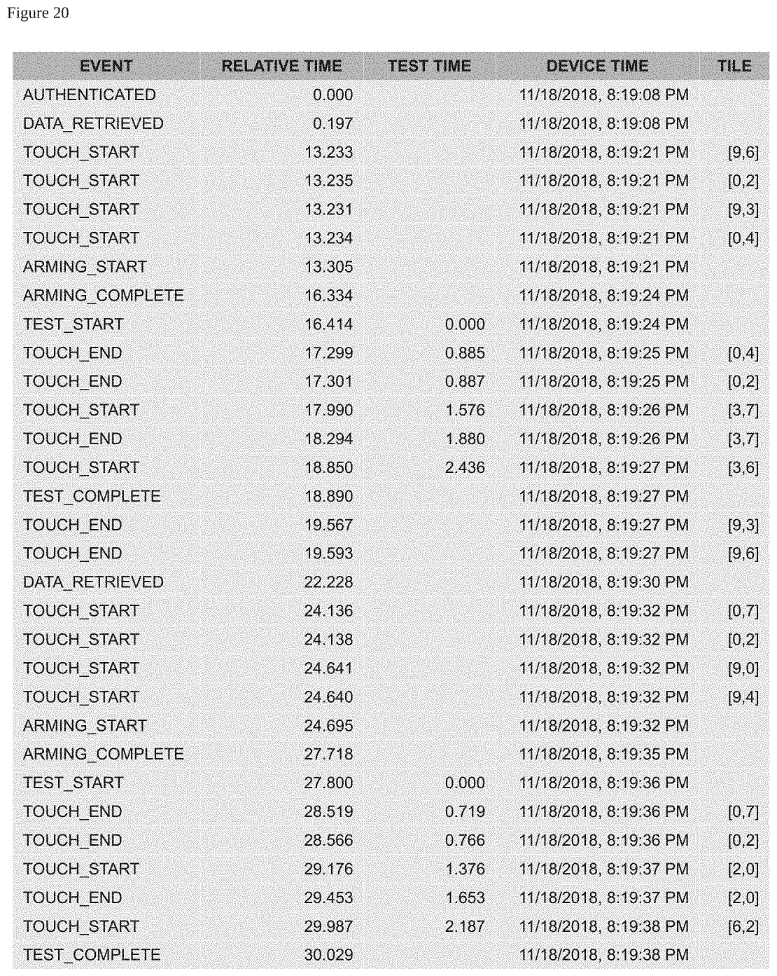

In certain embodiments, the CE system may capture a number of biometrics during one or more testing sessions, including a user's individual test completion times. The session timeline dataset may therefore represent the entirety of all data captured during the user's interactive session. For example, the session timeline may include all verified (e.g., via a debounce algorithm) touch start and touch end events, with corresponding absolute time stamp, relative time stamp and tile coordinate. FIG. 20 shows an exemplary timeline associated with the provision by the CE system of two discrete tests. In addition to the information depicted, in certain embodiments the session timeline may further include one or more of the following information:
1. Screen-pixel coordinates for touch events
2. System operations, including:
   a. user authentication completed
   b. data retrieved
   c. arming sequence initiated
   d. arming sequence terminated
   e. arming sequence completed
   f. test initiated
   g. test terminated
   h. test completed
3. Video or other recording of the users head, face and eye movements
   a. optional on-device eye tracking calculates screen gaze coordinates

For certain embodiments in which the captured timeline dataset includes user video, the CE system may synchronize timeline events with one or more encoded video frames in order to correlate user head, face and eye movements with specific session events. Additionally, the CE system may locally or remotely perform video frame analysis to calculate user gaze destination coordinates on the tablet screen.

In addition, the CE system may in certain embodiments analyze session timeline video to determine a user's distance from the display screen during a testing session or individual test. The CE system may perform such distance analysis during the course of the user interaction, either continuously or at specific time intervals. The tablet may reset or abort the arming and/or testing sequences if the CE system determines that the user's face is too close or too far from the display screen.

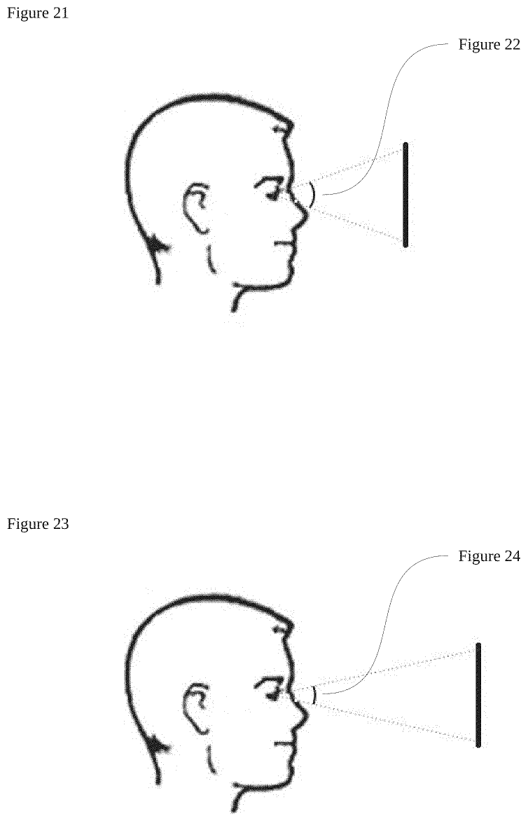

It is well known that a majority of visual recognition occurs within 15° of the user's line of sight. To that end, in certain embodiments the CE system may instruct the user to arrange the display screen at a sufficient distance from the user's eyes to ensure that the entirety of the display screen remains within this field of view. For example, the tablet in FIG. 21 is located too close to the user's face. As a result, the tablet screen content fills 42° of the user's maximum 30° field of view, as shown as FIG. 22. In contrast, tablet screen in FIG. 23 is located an acceptable distance from the user, filling only 28° of the user's field of view, as identified by FIG. 24. In certain embodiments, such distance analysis may be performed locally or remotely, for research or other purposes.

### 6. Interactive Session Flow

In various embodiments, a CE system testing session for a user may comprise one or more of the following distinct user interfaces:
1. Authentication interface
2. QR code camera scanner interface
3. Instruction interface
4. Testing interface

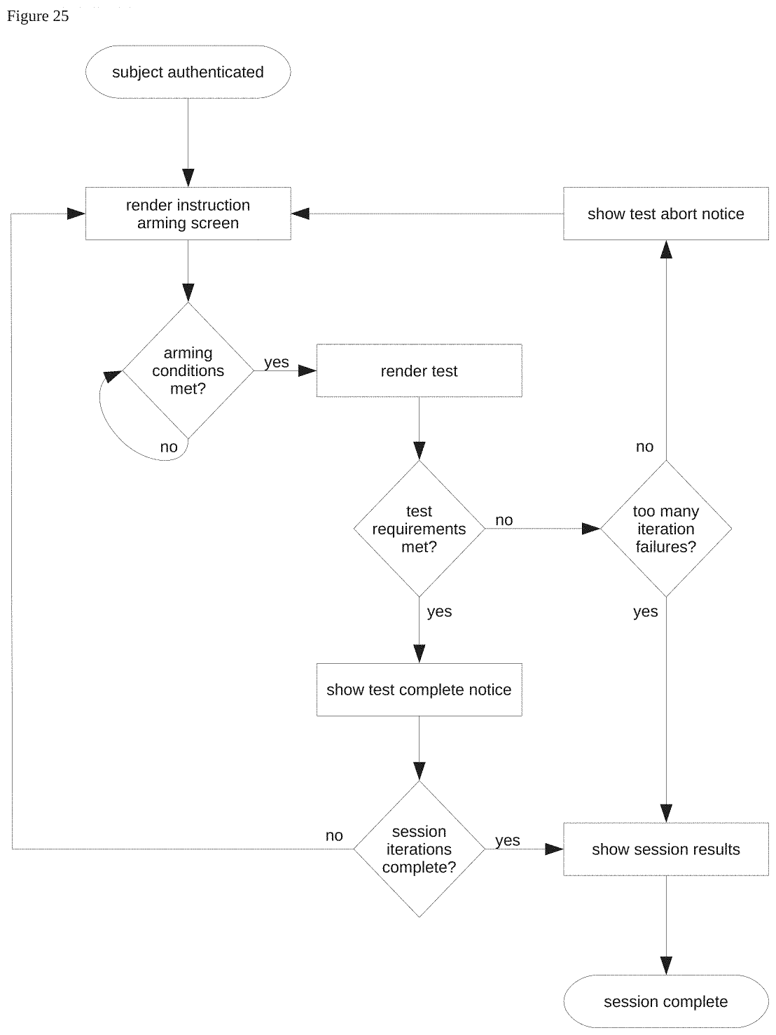

FIG. 25 depicts an exemplary session flow for an authenticated user utilizing an embodiment of the CE system during a single testing session. The instruction screen presents the test instructions to the user and provides interactive elements to initiate the arming sequence. The tablet application presents a standard notice dialog to the user at the end of each test displaying the completion time if the test is completed successfully, and the reason for abort if the test is not completed successfully.

The CE application may provide a notification to the user (e.g., an audio, visual, audiovisual, haptic, or other notification) upon completion of a CE testing session. In addition, the CE system may in certain embodiments indicate the quantity of tests completed successfully with a notice that the session has ended.

In certain embodiments, the locally executing CE application may communicate with the remote server to validate user credentials. If the credentials are valid, the remote server returns session and test parameters for one or more tests for the user to complete. Session parameters may include, as non-limiting examples, details such as background and foreground colors, number of tests to administer, test failure limits, requirements for face to screen distance, and tablet movement thresholds. Test parameters may in certain embodiments include some or all information needed to render a corresponding test, including: test instructions, arming icon images, arming icon positions, arming time, arming flash timings, test time limits, FIG. 16 images, target image(s), target image presentation mode, and FIG. 17 images.

In certain embodiments, the locally executing CE application may communicate with the remote server at various times (such as during or immediately following testing) to submit test results and/or retrieve additional tests to be administered to the user.

### 7. Test Classifications

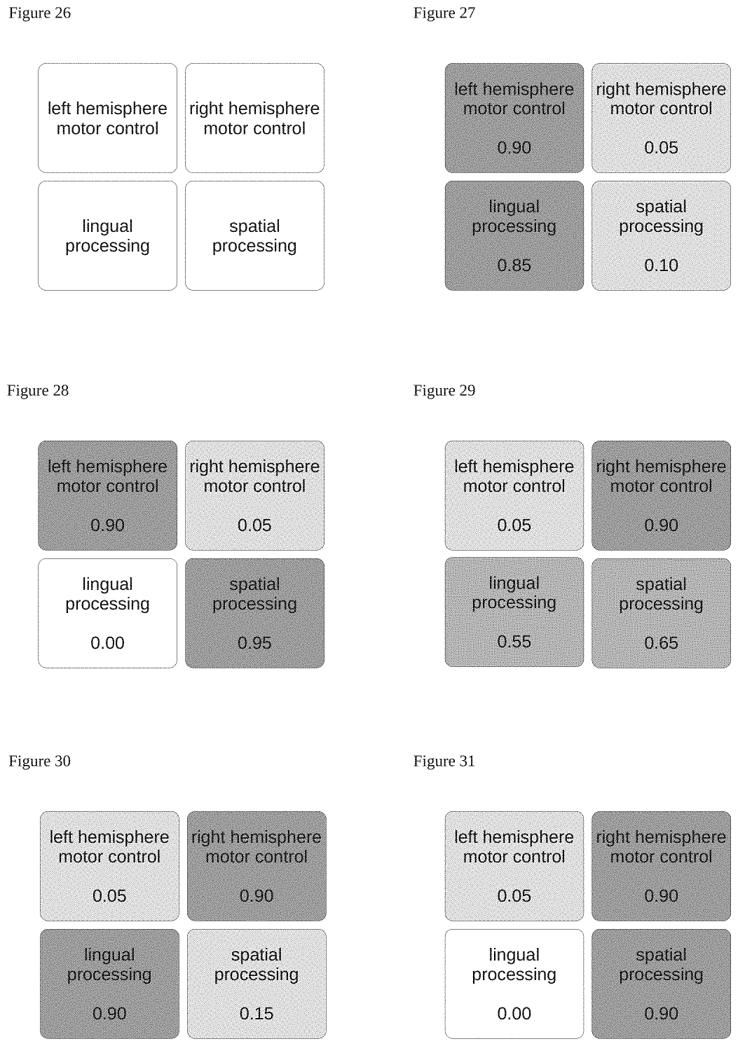

In at least one embodiment, the CE system may be configured to measure four cognitive processes: left brain motor control, right brain motor control, lingual processing and spatial processing. These cognitive processes are presented as a matrix in FIG. 26. However, additional embodiments of the cognition evaluation system may be configured to test for any quantity and variety of cognitive processes. Consequently, the size of the corresponding cognition classification matrix may vary significantly from the depicted 2 by 2 matrix, depending on the embodiment and desired use case scenario.

Cognition tests may be manually configured or may be automatically generated by the CE system to conform to specified criteria. Each test may require the user to successfully complete a plurality of cognitive processes. Tests are grouped into classes according to the cognitive processing they are configured to measure. Test classifications may be further differentiated by a measure of reliance on each of the respective cognitive processes that member tests require. In short, each class of tests empirically measures a particular combination of cognitive processes.

For example, FIG. 27 shows an embodiment of a CE system test class for which users utilize their right hand to select a target image that contains a typed word from one of the tile positions in FIG. 16, and then select the corresponding word from one of the tile positions in FIG. 17. The weights indicate a threshold dependence of cognitive processing corresponding to each area in the cognitive processes matrix. The user uses their left brain to control their right hand when selecting screen tile positions. However, the user may also be required to maintain the position of their left hand on the screen for the duration of the test. Consequently, the motor control processing measured by the test is split unevenly between the two cerebral hemispheres, with more processing being associated with the left-hemisphere responsible for the mobile right hand. The user is further required to perform significant lingual processing to make sense of the words and their meanings, but only nominal spatial processing for selecting the correct tiles positions on the screen.

A second example identifies another test class as FIG. 28. Tests in this class may require the user to use their right hand to select a target and complementary image that comprise of perspective line art drawings of simple geometric shapes. As in FIG. 27, the user motor control relies heavily on the left cerebral hemisphere. However, the requisite image comprehension along with the image positions on the screen are dependent solely on the user's spatial processes.

A third example, identified by FIG. 29, references yet another test class that requires the user to use their left hand to select a target and complementary image that contains both lingual and perspective line art elements. Correspondingly, this class is defined by a majority reliance on the right cerebral hemisphere with processing requirements distributed evenly between the lingual and spatial functions.

Tests within each class may vary significantly so long as their cognitive processing requirements reasonably match the relative processing weights that define the class.

In one embodiment, the CE system may determine to populate each test class with a wide variety of tests to lessen the impact of the user's memory on test results. To that end, the CE system may therefore vary numerous test variables including image content, image locations, screen background color, debounce timings, along with the arming sequence and even test instructions.

In certain embodiments, the CE system may target specific aspects of the user's memory by determining to vary a significant plurality or all test parameters within the test class with the exception of the desired cognitive characteristic being evaluated. In one example of this embodiment, the CE system may request that a user complete a large quantity of discrete tests (e.g., 500) during the course of a year. Of these tests, a relatively small proportion (e.g., 25) may belong to a single test class that evaluates pattern recall and left hemisphere muscle memory. In such an embodiment, the CE system may autonomously generate tests in this single class to meet the following criteria:
1. user is required to use their right hand to complete the test
2. user's left hand is required to maintain contact with the arming position for the duration of the test
3. the target image is always located at the tile position identified by FIG. 32
4. the complementary image is always located at the tile position identified by FIG. 33
5. all other complementary image candidates are displayed at positions indicated by FIG. 34
6. aside from the positions indicated by FIG. 33 and FIG. 34, no other tile positions indicated by FIG. 17 are populated with an image

All other test characteristics in this particular class may be varied significantly. Between each test in this class:
1. placement of target tile candidates in FIG. 16 excluding FIG. 32 is randomized
2. content of the target image is randomized between test
3. content of the complementary image is randomized (but maintains relationship to target image)
4. content and position of all target image candidates (that are not the target image) in FIG. 16 is randomized
5. the arming positions for the right and left hands are randomized

Typically, a user's completion time for this class of tests may improve over the course of the year, until it reaches a steady-state equilibrium with a nominal standard deviation. Given that the user continues tests of this class with maintenance-frequency regularity, a significant increase in completion time for this test class without comparable increases in other test classes indicates an impairment in user cognition as it relates to memory retrieval performance.

### 8. Test Result Analysis

In certain embodiments, client computing devices of the CE system may utilize an encrypted Transport Layer Security (TLS) or comparable protocol to communicate with a CE system server via one or more public or private computer networks, such as the Internet or other computer networks. In turn, the CE server may communicate with a plurality of such client computing devices to authenticate user credentials, distribute cognition tests, and receive session timeline data for analysis and archival. In certain embodiments, the CE server may comprise one or more physical or virtual computers operating as a cluster for increased security, scalability, and reliability. In such cluster configurations, individual computers may perform specialized tasks including authentication, message queuing, data storage or analytics. Likewise, resource intensive tasks such as data storage or analytics may be distributed across multiple computers to improve availability.

In at least one embodiment, the CE server may be configured to distribute only one class of cognition tests. It receives session timeline data for a user and parses it to obtain the completion time for the administered tests. Test timeline information for two exemplary sample tests are provided in the TEST TIME column of FIG. 20. The CE system would extract the following two timing values from the sample FIG. 20 session timeline: 2.436 and 2.187. The CE system may integrate these extracted test completion times with all previously collected completion times to maintain a running average and/or standard deviation for each user. Taken together, the CE system may determine to utilize these two calculated metrics as the user's baseline profile.

To determine whether a user's recent test metrics fit within their baseline norms, the CE system may use the following formula to calculate the upper completion time threshold:

[symbol]

### Where:

[symbol] =baseline profile max time threshold;
[symbol] =user average completion time;
[symbol] =standard deviation of user average completion time; and
[symbol] =a defined constant for the standard deviation threshold.

The CE system may then compare completion times associated with current session tests against the calculated [symbol] value. A test completion time greater than [symbol] indicates that the user is not within the their baseline norms and may be cognitively impaired.

The [symbol] value determines how far the user test score may be above their average, in terms of the user's standard deviation, before the user is considered to be outside their baseline norms. It is reasonable to expect that the user completion time average will reach a steady state and cease to improve. It is reasonable that the standard deviation of the user completion times will also reach a steady state and cease to decrease. In certain embodiments, a client or user definition of [symbol] is considered unreasonable; in contrast, a reasonable range for [symbol] may be considered to include values of (1, 5].

In certain embodiments in which the CE system administers a plurality of test classes, the CE system may determine to track a discrete baseline profile for each user for each test class. In this way, a user who has been administered a plurality of tests from ten different classes will have ten discrete baseline profiles, one for each test class. The CE system may consider the aggregate of a user's baseline profiles across all test classes to constitute the user's biometric fingerprint.

### 9. Identification of Impairment Source

If a user experiences a decrease in one of the cognitive processes being evaluated by a particular test, the user's completion time for that test will increase.

When the CE system identifies an increase in a user's completion time with respect to a particular test class, the CE system may determine to administer additional tests of the same class in order to confirm the initial results. Further, the CE system may determine to administer additional tests from other classes in order to evaluate varying combinations of cognitive processes that intersect the initial class combination. This iterative approach may allow the CE system to isolate the specific cognitive impairment(s) being experienced by the user.

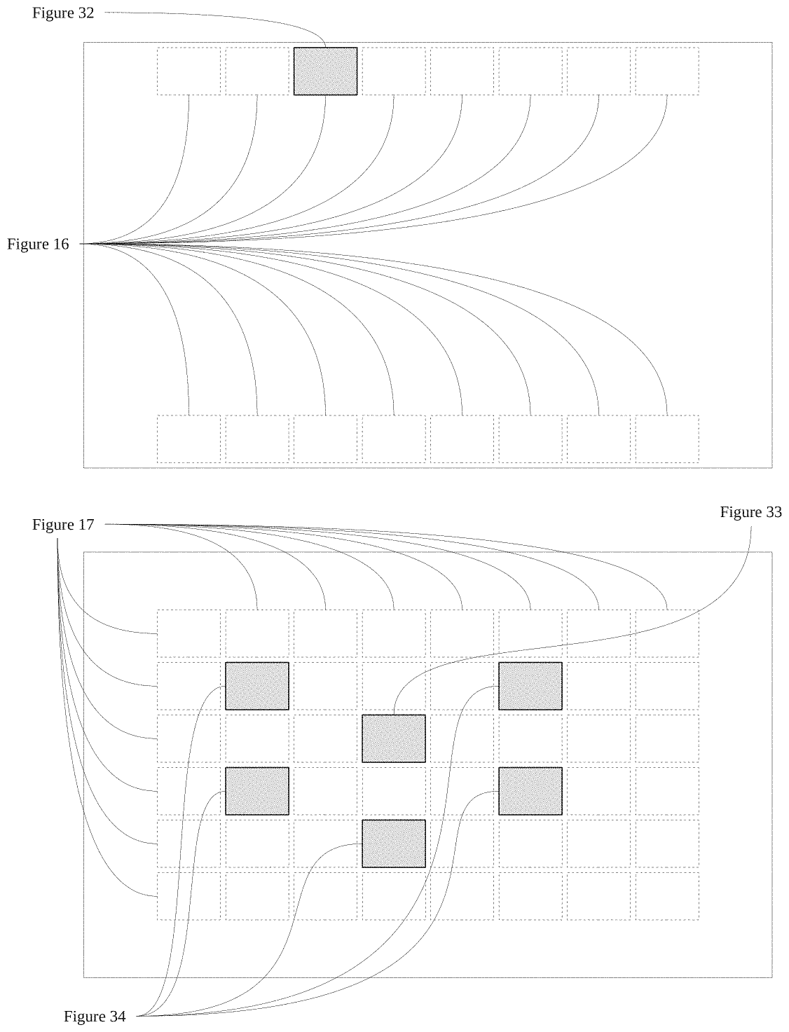

In one example, a user's test completion times from a test that belongs to the FIG. 29 class may far exceed their baseline norms and thereby indicate some form of impairment. To identify the specific impairment, the CE system may determine to administer additional tests from other classes, such as those depicted by FIG. 27, FIG. 28, FIG. 30 and FIG. 31. In this example, the CE system may determine that the user's test completion times for the FIG. 27 and FIG. 30 classes are within their baseline norms, but that the test times for the FIG. 28, FIG. 29 and FIG. 31 classes are significantly above their baseline norms.
FIG. 27 results—2.790 seconds—normal (average 2.685) [symbol]
FIG. 28 results—5.370 seconds—impaired (average 2.579) [symbol]
FIG. 29 results—4.982 seconds—impaired (average 2.616) [symbol]
FIG. 30 results—2.831 seconds—normal (average 2.714) [symbol]
FIG. 31 results—5.245 seconds—impaired (average 2.591)

[symbol]

For each impaired signal, the CE system may calculate the residual sum of squares for each of the four cognitive areas from FIG. 26, such as to identify a cognitive area that provides the best fit (lowest RSS) for that signal.

[symbol]

### Where:

i is the test class, such as those depicted by FIG. 27, FIG. 28, FIG. 29, FIG. 30 or FIG. 31;
weightᵢ is the weight of the cognitive area from the respective class;
overage_{impaired} is the time result overage from the impaired signal class being evaluated; and
overageᵢ is the time result overage from the respective class.

In certain embodiments, the CE system may calculate the residual sum of squares for each of the four cognitive areas from FIG. 26 using overage timing for a selected impaired signal, such as to identify which cognitive area best fits the selected impaired timing signal.

As one example, a result signal corresponding to FIG. 28 may be applied to the left hemisphere motor control of all classes:
FIG. 27 expected: 0.90*2.791=2.5119 measured: 0.90*0.105=0.0945
FIG. 28 expected: 0.90*2.791=2.5119 measured: 0.90*2.791=2.5119
FIG. 29 expected: 0.05*2.791=0.13955 measured:0.05*2.366=0.1183
FIG. 30 expected: 0.05*2.791=0.13955 measured: 0.05*0.117=0.00585
FIG. 29 expected: 0.05*2.791=0.13955 measured: 0.05*2.654=0.1327
   The resulting RSS is equal to:

[symbol]

### RSS=5.862

As another example, a FIG. 28 result signal may be applied to the right hemisphere motor control of all classes:
FIG. 27 expected: 0.05*2.791=0.13955 measured: 0.05*0.105=0.00525
FIG. 28 expected: 0.05*2.791=0.13955 measured: 0.05*2.791=0.13955
FIG. 29 expected: 0.90*2.791=2.5119 measured: 0.90*2.366=2.1294
FIG. 30 expected: 0.90*2.791=2.5119 measured: 0.90*0.117=0.1053
FIG. 29 expected: 0.90*2.791=2.5119 measured: 0.90*2.654=2.3886
   The resulting RSS is equal to:

[symbol]

### RSS=5.971

As another example, a FIG. 28 result signal may be applied to the lingual processing of all classes:
FIG. 27 expected: 0.85*2.791=2.37235 measured: 0.85*0.105=0.08925
FIG. 28 expected: 0.00*2.791=0.000 measured: 0.00*2.791=0.000
FIG. 29 expected: 0.55*2.791=1.53505 measured: 0.55 * 2.366=1.3013
FIG. 30 expected: 0.90*2.791=2.5119 measured: 0.90*0.117=0.1053
FIG. 29 expected: 0.00*2.791=0.000 measured: 0.00*2.654=0.000

[symbol]

### RSS=11.059

As another example, a FIG. 28 result signal may be applied to the spatial processing of all classes:
FIG. 27 expected: 0.10*2.791=0.2791 measured: 0.10*0.105=0.0105
FIG. 28 expected: 0.95*2.791=2.65145 measured: 0.95*2.791=2.65145
FIG. 29 expected: 0.65*2.791=1.81415 measured: 0.65*2.366=1.5379
FIG. 30 expected: 0.15*2.791=0.41865 measured: 0.15*0.117=0.01755
FIG. 29 expected: 0.90*2.791=2.5119 measured: 0.90*2.654=2.3886

[symbol]

### RSS=0.325

In certain embodiments, the CE system may therefore determine that a cognitive area with the lowest RSS is the closest match to the measured timing signal provided by FIG. 28. For example, the CE system may determine that in the depicted examples, the spatial processing area best fits the FIG. 28 timing signals, as it has the lowest RSS.

The CE system may repeat the process for the remaining impaired signals in this example: FIG. 29 and FIG. 31.

The FIG. 29 result signal is applied to the left hemisphere motor control of all classes; to the right hemisphere motor control of all classes; to the lingual processing of all classes; and to the spatial processing of all classes. The CE system may thereby determine that the cognitive area with the lowest sum is the closest match to the measured timing signal provided by FIG. 29, and therefore the most likely to be impaired.
FIG. 29 result signal RSS for left hemisphere motor control:

[symbol]

### RSS=4.230

FIG. 29 result signal RSS for right hemisphere motor control:

[symbol]

### RSS=4.177

FIG. 29 result signal RSS for lingual processing:

[symbol]

### RSS=7.790

FIG. 29 result signal RSS for spatial processing:

[symbol]

### RSS=0.395

The CE system may therefore determine that the spatial processing area best fits the FIG. 29 timing signals, at is has the lowest RSS. The result of the analysis for FIG. 29 matches the result for FIG. 28.

Additionally, the CE system may determine to apply the FIG. 31 result signal to the left hemisphere motor control of all classes; to the right hemisphere motor control of all classes; to the lingual processing of all classes; and to the spatial processing of all classes. The result of the analysis for FIG. 31 may typically match the result for FIG. 28 and FIG. 29, such that the cognitive area with the lowest sum is the closest match to the measured timing signal provided by FIG. 31 and is the most likely to be impaired.
FIG. 31 result signal RSS for left hemisphere motor control:

[symbol]

### RSS=5.294

FIG. 31 result signal RSS for right hemisphere motor control:

[symbol]

### RSS=5.297

FIG. 31 result signal RSS for lingual processing:

[symbol]

### RSS=9.933

FIG. 31 result signal RSS for spatial processing:

[symbol]

### RSS=0.262

In this example, the CE system may determine that the spatial processing area best fits the FIG. 31 timing signals, at is has the lowest sum. The result of the analysis for FIG. 31 matches the results for FIG. 28 and FIG. 29.

The CE system may further determine a confidence level for one or more cognitive evaluation tests, such as may be indicated by a degree to which the analysis for multiple impaired response signals correspond.

In this manner, the CE system may quickly evaluate a user by administering a nominal number of tests, each of which may enable the CE system to detect a specific plurality of potential impairment conditions. Moreover, responsive to identifying one or more sub-optimal results, the CE system may initiate the administration of one or more additional tests to further confirm and isolate potentially impaired cognition areas.

In certain embodiments, the CE system may determine to perform various post-validation operations regarding one or more test results to ensure that the administered test conforms to specified criteria, such as to satisfy a designated classification for the administered test. To facilitate this, the CE system may store test parameters for some or all tests administered in each testing session in addition to the test results for that testing session.

The various embodiments described above can be combined to provide further embodiments. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A cognitive evaluation system, comprising:
one or more processors;
a display device;
a plurality of user input actuators; and
a computer-readable storage medium having instructions stored thereon that, when executed by the one or more processors, cause the system to:
provide a plurality of cognitive evaluation tests to one or more users over a first period of time;
determine baseline cognitive evaluation information for at least one user of the one or more users based at least in part on test results from the plurality of cognitive evaluation tests;
administer one or more additional cognitive evaluation tests to the at least one user via the display and the plurality of user input actuators;
identify, based at least in part on test results from the one or more additional cognitive evaluation tests, a cognitive impairment condition of the at least one user.

2. The system of claim 1, wherein the display device and at least some of the plurality of user input actuators comprise a touch display.

3. The system of claim 1, further comprising one or more cognitive evaluation server computers, wherein at least one of the one or more cognitive evaluation server computers performs one or more operations to determine the baseline cognitive evaluation information for the at least one user.

4. The system of claim 1, wherein to determine the baseline cognitive evaluation information for the at least one user includes to determine the baseline cognitive evaluation information based on multiple cognitive evaluation tests administered to the at least one user over a first time period.

5. The system of claim 1, wherein the baseline cognitive evaluation information for the at least one user is based at least in part on test results for one or more users other than the at least one user.

6. The system of claim 1, wherein to administer the one or more additional cognitive evaluation tests includes to administer a plurality of additional cognitive evaluation tests, such that each of the plurality of additional cognitive evaluation tests measures a combination of one or more distinct cognitive processes.

7. The system of claim 1, wherein to administer the one or more additional cognitive evaluation tests includes requiring the at least one user to perform one or more manual operations with respect to the plurality of user input actuators in a timed manner.

8. The system of claim 7, wherein the baseline cognitive evaluation information for the at least one user is based at least in part on one or more response times associated with the one or more manual operations.
